(19) United States
(12) Patent Application Publication
(10) Pub. No.: US 2011/0270133 A1
(43) Pub. Date: Nov. 3, 2011

(54) Steson's Algorithm

(76) Inventor: Stellan Eriksson, Stockholm (SE)

(21) Appl. No.: 12/782,638

(22) Filed: May 18, 2010

Related U.S. Application Data
(60) Provisional application No. 61/329,395, filed on Apr. 29, 2010.

(51) Int. Cl. A61B 5/103

(52) U.S. Cl. 600/587

(57) ABSTRACT

A method of electing a patient suffering from low back pain (LBP) for the treatment of a physiotherapist or to undergo an imaging process for further analysis of an underlying complication, said method comprises a predetermined sequence of physical examinations.

### RELATED APPLICATION

The present application claims priority under 35 U.S.C. §119 of U.S. Application No. 61/329,395 filed Apr. 29, 2010.

### TECHNICAL FIELD

The present invention relates to a method of electing a patient suffering from low back pain (LBP) for the treatment of a physiotherapist or to undergo an imaging process for further analysis of an underlying complication.

### BACKGROUND OF INVENTION

Mechanical low back pain (LBP) produces major societal, industrial and personal problems, resulting in substantial annual health care costs and sick-leaves.

In the US the costs has increased from 11.4 billion US Dollars 1989 to 100-150 billion US dollars 2009 (E-medicine from WebbMD, Mechanical low back pain, Hills EC, updated Mar. 24, 2010). Besides the economical facts, there is a personal tragedy with pain and physical disability that will be prolonged if not properly handled.

Lumbar muscle function is considered to be an important component of chronic lower back pain (LBP). LBP is a common symptom of musculoskeletal disorder. Complementary studies have documented compromised muscle function in patients with LBP. Although the mechanism associating muscle insufficiency to LBP is not clearly understood, it is commonly held that the passive tissues of the spine are increasingly stressed with increasing functional muscle insufficiency. Only a small percentage of LBP complaints can be diagnosed definitely because current techniques are effective only for diagnosis of LBP associated with damage or abnormality of the skeleton. However, a substantial percentage of these complaints can't be diagnosed because existing techniques are ineffective for diagnosis of LBP associated with muscular dysfunction.

The term low back pain is used to describe the condition of up to 85% of patients for whom a specific diagnosis is said to be impossible.

Muscle aches, muscle sprains, tendonitis, sacroiliac and low back strain, lumbago, mechanical LBP, and lumbar strain are some of the currently used diagnosis in clinical use.

The high incidence of back injury among people cost the society a lot of money each year partly because there is no fast and safe diagnosis methods and the physicians do not have better methods than sending the patient for physical diagnosis such as myelography, computed tomography (CT), and magnetic resonance imaging (MRI) that in addition to the costs also expose many of the patients for unnecessary radiation.

Earlier lack of evidence-based research for idiopathic low back pain has focused on two opposing opinions; a tissue derived pain or a nonspecific one.

The pain is of neurologic origin and pressure from muscle and skeleton on nerve structures exert the prime action.

On the other hand, disclosure of medical conditions presenting as LBP is of importance.

It is of great importance to improve the methods for diagnosing LBP since a major diagnostic problem with LBP is that many anatomic abnormalities seen on imaging tests are common in healthy individuals. The abnormalities often results from age-related degenerative changes, which begin to appear early in the adulthood and are in some ways analogous to gray hair and wrinkles (Richard A. Deyo. Diagnostic evaluation of LBP. American Medical Association. Arch intern med. Vol. 162, 2002).

Conventional therapeutic measures have been regarded as obsolete and a more broad view of the underlying mechanisms behind back pain is taken into account.

Moreover, the physicians have no other choice than put patients on the sick list when the patient has pain.

In priori art the Veteran Health Administration and the Department of Defense (VHA/DoD) has published a clinical practice guideline for the management of low back pain or sciatica (Management of low back pain or sciatica. Veteran Health Administration, Department of Defense. May, 1999). This document presents an algorithm of 22 steps for the diagnosis of LBP. However the method does not include checkpoints for an efficient investigation and includes many meetings for the patient.

Most diagnosis methods available today which assess muscle deficiencies are either nonobjective or they lack rigorous clinical validation and reliability. The guidelines circulated for clinical use fail to offer clinical algorithms for the various non-disk-related problems that might cause the patient's pain.

There is still a need for a reliable methodology to perform an early discrimination of patients suffering from LBP who are treatable by physiotherapy and those patients who are suffering from other conditions.

The object of this invention, therefore, is to provide an improved method for analyzing muscle fatigue associated with LBP and also reduce the costs for unnecessary expensive and time consuming investigations.

### SUMMARY OF INVENTION

This method describes a physical examination procedure in perpendicular planes that will explain the common pathology of low back pain, LBP.

An object of the present invention is to provide an improved method based on an algorithm for diagnosis of lower back pain (LBP).

It is an object of the present invention to obviate at least some of the disadvantages in the prior art and provide a more effective and reliable method for the elucidation of lumbago.

In a first aspect there is provided a method of electing a patient suffering from low back pain (LBP) for the treatment of a physiotherapist or to undergo to an imaging process for further analysis of an underlying complication.

In a second aspect the use of the method is provided to electing a patient suffering from low back pain (LBP) for the treatment of a physiotherapist or to undergo to an imaging process for further analysis of an underlying complication.

The provided method comprises a predetermined sequence of physical examinations comprising the steps of:

a) examining the gait and posture
b) examining the hip height symmetry
c) examining the crista iliaca posterior inferior (CIPI) Level
d) examining the malleoli level
e) Performing Patricks' test
f) examining the pubic level
the method is conducted by performing each of the examination steps a) to f) until a physical examination of such a step confirms a dysfunctional cause explaining the LBP and electing such patient to physiotherapy. A patient who is not observed to exhibit any of dysfunctionalities discernable from the examination steps a) to f) is elected for an imaging process.

The method comprises a predetermined sequence of physical examinations comprising examination steps, wherein each step is performed in the particular sequence according to FIG. 1.

The consequence of when one of the steps in the algorithm do not pass the physical examination, is that underlying cause of LBP is a muscular complication that can be treated by a physiotherapist.

On the contrary, if all investigation steps in the algorithm pass and is considered to be normal, the cause for the LBP is probably not muscular but more likely to involve complications in the lumbar vertebrae.

In the context of the present investigation, the tests in the algorithm are described in the best mode of performance, some patients may have difficulties to comply with them e.g. to flex the knee to ninety degrees, therefore, the angles and movements may differ depending on the patient's possibility to move their body.

One advantage of the method is the fast (2-5 minutes) and reliable election of patients that suffer from LBP for the treatment by a physiotherapist or if they need further examinations by imaging methods.

Another advantage of the invention is that many people will not have to undergo costly and unessential investigations such as NMR, X-Ray, CT etc that includes harmful radiation to the patient. In addition, the sick-listing of patients with low back pain will be lower since they get correct treatment by a professional physiotherapist or similar.

Another advantage is that the sick-listing of patients during a back-pain investigation may be shortened or even not necessary when excluding other causes than muscular to the back pain.

A further advantage is the lowered cost for the society for unnecessary investigations and sick-leaves, and for the patient it results in lower radiation due to the unnecessary investigations and also less time wasted on investigations.

Further aspects and embodiments are defined in the appended claims, which are specifically incorporated herein by reference.

### BRIEF DESCRIPTION OF DRAWINGS

The invention is now described, by way of example, with reference to the accompanying drawings, in which:

FIG. 1 shows an algorithm for electing patients suffering from low back pain to physiotherapy or further investigation by imaging methods.

FIG. 2 shows the mobilization examination test that discriminates muscular causes in a positive Patricks' test.

FIG. 3 shows the mobilization examination test that discriminates muscular causes in a positive pubic upper level examination.

### DETAILED DESCRIPTION

In the following part, a detailed description of the invention will follow, but before doing that it must be clear that the tests described here may somewhat be modified to agree with the patient's ability to move.

In the context of the present invention the examination tests are performed by a health care provider, e.g., a physiotherapist, chiropractor, napraphat or a physician.

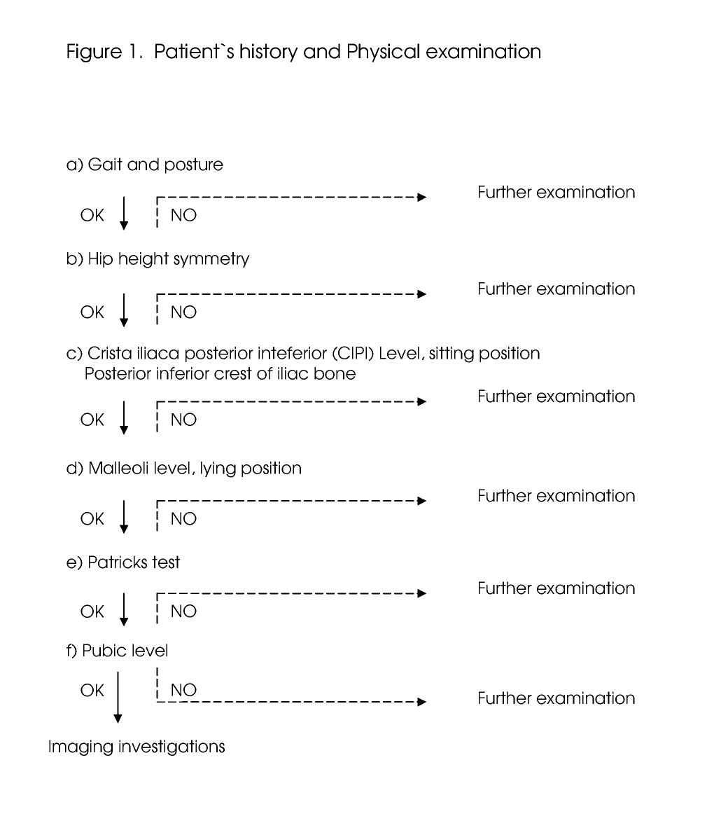

FIG. 1 shows the present invention in the form of an algorithm. The algorithm is used for electing a patient suffering from LBP for the treatment of a physiotherapist or if further investigations by a physician are needed. If muscular causes are identified in any investigation step of the method presented in FIG. 1 the patient will be sent to a physiotherapist (or similar) for further investigations or treatment. If no muscular causes are identified by the investigation steps comprised in the algorithm, the patient must undergo further investigations by a physician that may send the patient for further investigations such as imaging methods.

The algorithm presents the physiological examination of the patient step by step and in also what order they should be investigated. The result of each examination step (step a) to f) in FIG. 1) has to be in the physiological normal range to pass the step i.e., show symmetry (OK in FIG. 1), to be allowed to go to the next examination step presented in the algorithm.

The first step involves an ocular investigation to determine whether or not the patients gait and posture (examination step a) in FIG. 1) is normal. If not, the cause to asymmetry must be investigated. Attempts to correct the failure by known methods to a person skilled in the art will also be performed. The patient may also improve the gait and posture by time. For example, if the cause to abnormal gait or posture is because of different lengths of the legs (i.e., skeleton-related) that may be the cause to the back pain and the length should be corrected by e.g. an insert in the shoe. But if the examiner can't identify any problems in step a and find that the hips are in asymmetry, it is most likely that the problems have a background in muscular dysfunction. Therefore, a physiotherapist should be contacted (dotted arrow in FIG. 1) and an unnecessary costly investigation is avoided. If the gait and posture seems to be normal, the next step is to investigate the symmetry of the hips (step b).

The symmetry of the hips is investigated by both an ocular and physical examination both in a standing and a sitting position, respectively. The back hands are pointing upward upon upper pelvic regions in a standing position. If an estimation of less than 1 cm is noticed in the cranio-caudal plane, go to step c. If there is an asymmetry and the hips is disturbed because of e.g. hip deformity/infection in the young or an accident try to correct the level of the hips with known methods and/or contact a physiotherapist for further investigation.

The next step (step c) is to investigate Crista Iliaca posterior inferior level (CIPI) (posterior inferior crest of iliac bone). First of all, the structure and function in a sitting position is examined. The structure is obtained by the level of the examiner's thumbs positioned below the bone prominences on persons in a sitting position. The functional component is obtained from the thumbs positioning, when the client is bending forward. It is very important to observe if the thumbs movement is congruent or not. Differences less than 0.5 cm will be identified. The rotation forward makes it possible to see a functional symmetry or asymmetry, which in turn can explain a virtual paradox later on. If the examination don't show any muscular abnormal conditions, go to examination step d. If the patient do not pass the muscular test, the patient should be sent for further examination by a physiotherapist or similar.

Next step is to investigate the symmetry of the malleoli level (step d). This is done when the patient is in a lying position. The examiner holds the thumbs preferentially positioned below the medial malleoli close to each other and determines whether the malleoli level show symmetry or not. If they show symmetry, the next examination step is performed. If not, the patient should be sent for further examination by a physiotherapist or similar.

The examination of the hip/pelvic zone is performed by outward rotation by using a modified Patrick's test (Step e) which is performed by supporting the heel below the knee level of the other leg, a slight pressure in- and upward on the knee is applied for about a second and then, an outward reverse movement (MET) of knees is done for the purpose to obtain an optimal movement that will discriminate the positive side with less mobility. The heel level may vary somewhat due to physical problems for the patient. Side discrimination, is a qualitative and quantitative test that obtains the side of pain(s) and degree of movement. If a difference in malleoli level was identified earlier, or from the symmetry test, next examination step should be performed i.e., a mobilization test of the affected side.

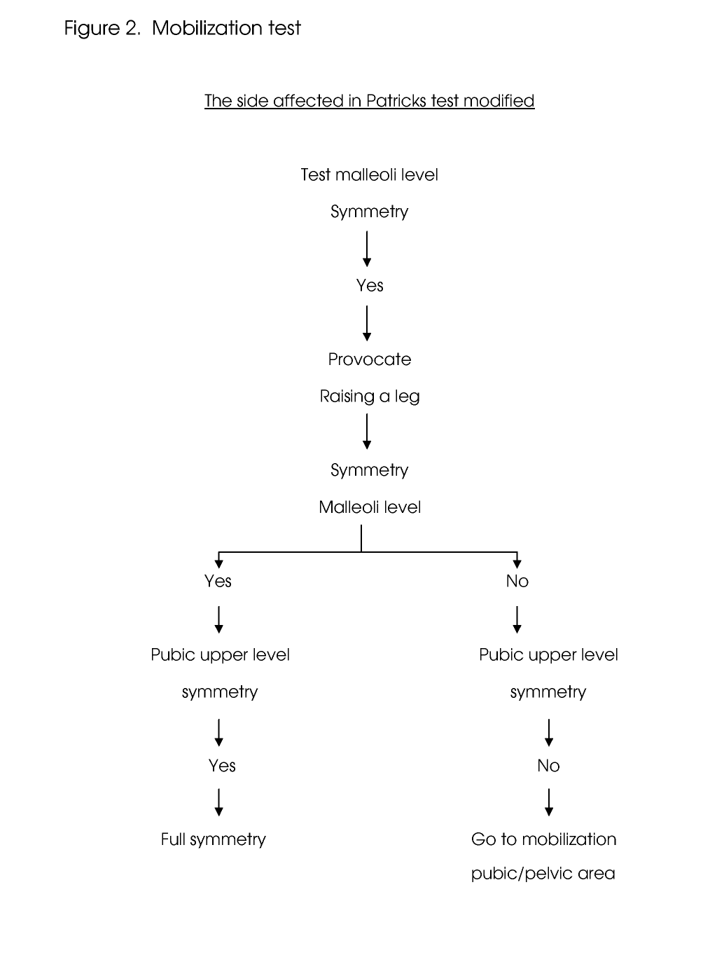

In the context of the present invention Patrick's test is performed by a health care provider to assess if the underlying cause of LBP is sacroiliitis (inflammation of the sacroiliac joint). The knee is flexed to ninety degrees on the affected side and the foot is rested on the unaffected knee. Holding the pelvis firm against the examination table, the affected-side knee is pushed towards the examination table, a maneuver which provides external rotation of the leg at the hip joint. If pain results, this is considered a positive Patrick's test and sacroiliitis is more likely. However, Patrick's test does not prove that sacroiliitis is causing the back pain, just increases the likelihood. Patrick's test is primarily useful in evaluating pathology of the hip joint. A positive test can be slightly indicative of a sacroiliac problem but not very reliably. A severely restricted and painful Patrick's test can be found in patients suffering from a degenerative disease of the hip or in traumatic injury to the hip. Patients having a positive outcome of Patricks' test are further subjected to the mobilization test, described in FIG. 2.

The mobilization test (FIG. 2) of the side affected by MET (muscular energy technique) as seen above and obtain symmetry by a slight pressure from the knee perpendicular to the horizontal level upward and then lift to half maximum of the affected pelvic side for some seconds. Then raise the pelves from the ground. The shoulders and the feet should still be touching the ground and the malleol levels should now be in symmetry, otherwise repeat the test. Provocate to see if asymmetry exists in other planes by raising one or two legs upwards. If asymmetry turns up in either CIPI or malleol level go back to step c) and step d) according to the present invention and try to regain the symmetry.

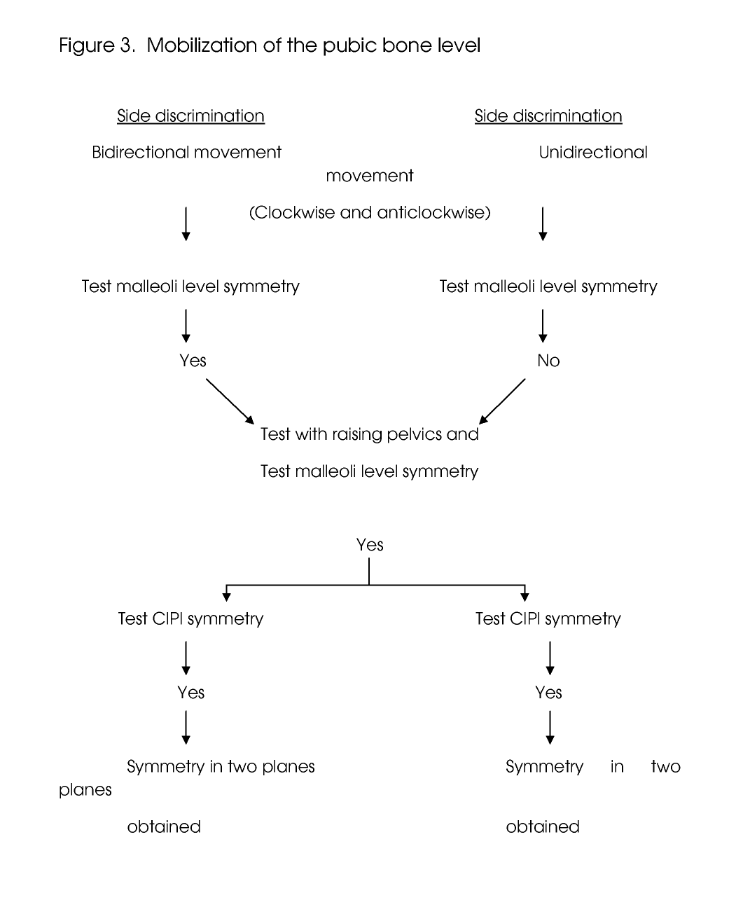

Pubic upper level is examined (step f) with two fingers wherein one finger is on respective pubic bone, discrepancy is measurable. Apply a MET tension in the part to be stretched and rotate the pelvic to loosen the muscular tension. Provocate again and control the hip movement in the same phase of investigation. Mobilize the side affected (positive in Patricks' test) positioned upward as described in FIG. 3. The pubic level, on the positive side, decides the route of rotation for symmetry of pelves after MET.

Test malleoli level, if discrepancy, raise the pelves from the ground and symmetry are obtained in malleoli and CIPI level and the two planes studied.

Another way in this last mobilization is to rotate both clockwise and anticlockwise, this will give symmetry without raising the pelves (FIG. 3).

Control of malleoli-, CIPI- and pubic levels should show symmetry, if not go back to the modified Patricks' test again and try to obtain expected symmetry.

It is of choice to use both the CIPI- and malleoli-tests for easier discrimination in obese or fractured individuals.

A skilled physiatrist will manage to analyze, treat and inform about LBP. The muscular derived affection is of a plastic nature and will easily revert into incongruence. If nothing else is defined, any terms and scientific terminology used herein are intended to have the meanings commonly understood by those of skill in the art to which this invention pertains.

1. A method of electing a patient suffering from low back pain (LBP) for the treatment of a physiotherapist or to undergo an imaging process for further analysis of an underlying complication, said method comprises a predetermined sequence of physical examinations comprising the steps of:
a) examining the gait and posture
b) examining the hip height symmetry
c) examining the crista iliaca posterior inferior (CIPI) Level
d) examining the malleoli level
e) Performing Patricks' test
f) examining the pubic level
wherein the method is conducted by performing each of the examination steps a) to f) until a physical examination of such a step confirms a dysfunctional cause explaining the LBP and electing such patient to physiotherapy; and
wherein a patient who is not observed to exhibit any of dysfunctionalities discernable from the examination steps a) to f) is elected for an imaging process.

2. A method of electing a patient suffering from low back pain (LBP) for the treatment of a physiotherapist or to undergo an imaging process for further analysis of an underlying complication, said method comprises a predetermined sequence of physical examinations comprising examination steps, wherein each examination step is performed in the particular sequence according to FIG. 1.
